(19) United States
(12) Patent Application Publication
(10) Pub. No.: US 2026/0240585 A1
(43) Pub. Date: Aug. 20, 2026

(54) SMOKE AND FLUID EVACUATION DEVICES AND SYSTEMS

(71) Applicant: Megadyne Medical Products, Inc., Draper, UT (US)

(72) Inventors: Paul R. Borgmeier, Salt Lake City, UT (US); Shawn K. Horner, Woods Cross, UT (US)

(21) Appl. No.: 19/058,351

(22) Filed: Feb. 20, 2025

(51) Int. Cl. A61B 18/00

(52) U.S. Cl. CPC A61B 18/00; A61B 2218/008

(57) ABSTRACT

A smoke evacuation may be configured to have a fluid evacuation device selectively joined thereto to provide both smoke and fluid evacuation capabilities. A combination smoke and fluid evacuation may include a smoke evacuation hose, a fluid evacuation hose, a wand and a switching device to selective switch fluid communication between the wand and the hoses for smoke evacuation or fluid evacuation.

### BACKGROUND

### 1. Technical Field

This disclosure relates to evacuation devices and systems for removing smoke and fluid from a surgical site or environment.

### 2. The Relevant Technology

As is known to those skilled in the art, modern surgical techniques typically employ radio frequency (RF) power to cut tissue and coagulate bleeding encountered in performing surgical procedures. Such electrosurgery is widely used and offers many advantages including the use of a single surgical instrument for both cutting and coagulation. A monopolar electrosurgical generator system has an active electrode, such as in the form of an electrosurgical instrument having a hand piece and a conductive electrode or tip, which is applied by the surgeon to the patient at the surgical site to perform surgery, and a return electrode to connect the patient back to the generator.

The electrode or tip of the electrosurgical instrument is small at the point of contact with the patient to produce an RF current with a high current density in order to produce a surgical effect of cutting or coagulating tissue through cauterization. The electrode or tip of the electrosurgical instrument communicates electrical energy to a target tissue of a patient to cut the tissue and/or cauterize blood vessels within and/or near the target tissue.

The cutting and cauterization by the electrosurgical instrument results in smoke released into the air that can be distracting, unpleasant, or otherwise undesirable. Many surgical environments that employ electrosurgical systems include an evacuation system that captures the resulting smoke and directs it through a filter and exhaust port, away from practitioners and/or patients. Smoke evacuation systems commonly include a suction wand connected to a vacuum device via tubing. The surgeon or assistant holds the suction wand close to the surgical site and the smoke is drawn into the suction wand and through the tubing.

However, using a separate smoke evacuation device is not ideal. Using a separate smoke evacuation device requires additional hands and instruments near the surgical site, which can obscure the surgeon’s view of the surgical site and reduce the room available around the surgical site for the surgeon to move. In an effort to remedy this, electrosurgical instrument and smoke evacuation combination devices have been developed. These combination devices often include a hand piece that can receive an electrode or tip for performing electrosurgical procedures. The hand piece is connected to a generator via a power cable to convey RF current to the electrode or tip. Additionally, a smoke evacuation hose is connected between the hand piece and a vacuum to draw smoke away from the surgical site.

While such combination devices may reduce the total number of individual devices and hands around the surgical site, they have their own drawbacks. For instance, the smoke evacuation hose can create drag or resistance to the movement of the hand piece. This drag or resistance can make it more difficult for the surgeon to control the movements of the electrode or tip, which can pose particular challenges when trying to perform delicate procedures. Additionally, the constant effort to overcome the drag or resistance can cause the surgeon’s hand to tire or become fatigued.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

Generally, the present disclosure relates to devices for evacuating smoke and fluid from surgical sites. For instance, in one embodiment, a smoke evacuation device comprises: a hub comprising an interior cavity, a port that is configured to provide selective access to the interior cavity, a distal opening, and a hose mount in fluid communication with the interior cavity; and a nozzle extending from the hub, the nozzle having a lumen extending therethrough that is in fluid communication with interior cavity via the distal opening in the hub, the nozzle comprising one or more openings in a distal end thereof, the port, interior cavity, and nozzle being configured to have a fluid evacuation device selectively positioned therethrough such that a portion of the fluid evacuation device extends out of the port and a portion of the fluid evacuation device extends out of the distal end of the nozzle.

According to other example embodiment, a combination smoke and fluid evacuation device for use during an electrosurgical procedure to evacuate smoke and fluid away from a surgical site comprises: a wand configured to be used to collect smoke and fluid from a surgical site; a smoke evacuation hose mount that is configured to be connected to a smoke evacuation hose; a fluid evacuation hose mount that is configured to be connected to a fluid evacuation hose; a switching component that is configured to enable selective fluid communication between the wand and alternately the smoke evacuation hose mount and the fluid evacuation hose mount.

This Brief Summary is provided to introduce a selection of concepts in a simplified form that are further described below in the Detailed Description. This Brief Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates an electrosurgical system according to one exemplary embodiment of the present disclosure.

FIG. 2A illustrates the smoke evacuation device according to one exemplary embodiment of the present disclosure that may be used with the electrosurgical system of FIG. 1.

FIG. 2B illustrates a cross-sectional view of the smoke evacuation device of FIG. 2A.

FIG. 3A illustrates the smoke evacuation device of FIGS. 2A-2B fitted with an optional fluid evacuation device.

FIG. 3B illustrates a cross-sectional view of the smoke and fluid evacuation devices of FIG. 3A.

FIG. 4A-4C illustrate distal ends of the smoke and fluid evacuation devices of FIGS. 2A-3B.

FIG. 5 illustrates a smoke and fluid evacuation device that may be used in or with the electrosurgical system of FIG. 1.

FIG. 6A illustrates a cross-sectional view of the smoke and fluid evacuation device of FIG. 5 in a fluid evacuation mode.

FIG. 6B illustrates a cross-sectional view of the smoke and fluid evacuation device of FIG. 5 in a smoke evacuation mode.

### DETAILED DESCRIPTION

The present disclosure relates to hand-held instruments that facilitate the removal of smoke and/or fluid from a surgical site.

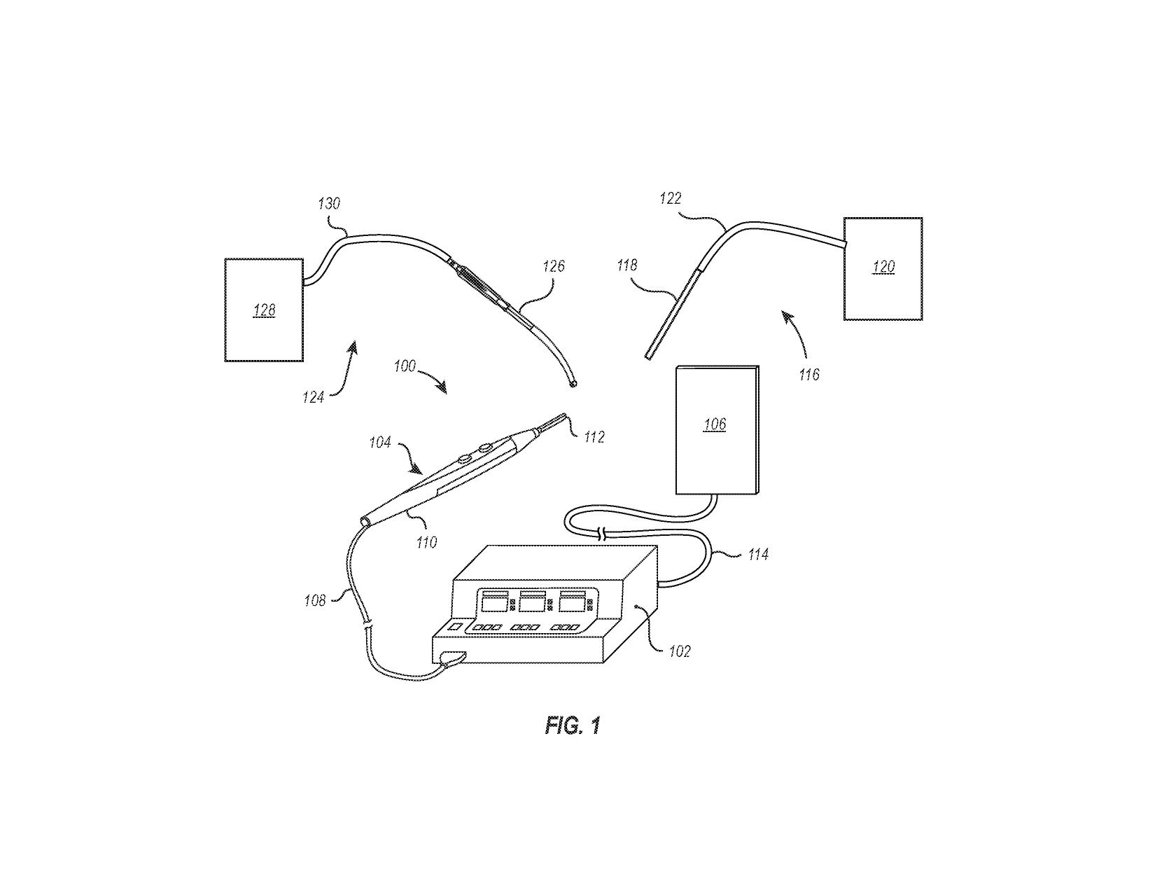

Referring to FIG. 1, a typical electrosurgical operating environment is illustrated. In FIG. 1, the electrosurgical operating environment includes an electrosurgical system 100, which includes a signal generator 102, an electrosurgical instrument 104, and a return electrode 106. The signal generator 102 is an RF wave generator that produces RF electrical energy and communicates the RF electrical energy to the electrosurgical instrument 104 via a cable 108. The electrosurgical instrument 104 includes a hand piece 110 and an electrode tip 112. The electrosurgical instrument 104 communicates the RF electrical energy to a patient to cut tissue and/or cauterize blood vessels of the patient’s body. Specifically, an electrical discharge is delivered from the electrode tip 112 to the patient in order to cause the heating of cellular matter of the patient that is in extremely close contact to the electrode tip 112. The heating takes place at an appropriately high temperature to allow the electrosurgical instrument 104 to be used to perform electrosurgery. The return electrode 106 and a cable 114 provide a return electrical path to the signal generator 102 for any excess charge dissipated into surrounding tissue of the patient's body.

The electrosurgical operating environment of FIG. 1 also includes a smoke evacuation system 116. The smoke evacuation system 116 includes a hand piece 118 that is connected to a pump 120 via a hose 122. The pump 120 may generate a negative pressure that is communicated to the hand piece 118 via a lumen in the hose 122. The hand piece 118 may include one or more openings that communicate the negative pressure to the surrounding environment in order to draw smoke into the hand piece 118. The smoke can be communicated from the hand piece 118, through the hose 122, and to the pump 120.

The electrosurgical operating environment of FIG. 1 also includes a fluid evacuation system 124. The fluid evacuation system 124 includes a hand piece 126 that is connected to a pump 128 via a hose 130. The pump 128 may generate a negative pressure that is communicated to the hand piece 126 via a lumen in the hose 130. The hand piece 126 may include one or more openings that communicate the negative pressure to the surrounding environment in order to draw fluid into the hand piece 126. The fluid can be communicated from the hand piece 126, through the hose 130, and to the pump 128.

During a typical electrosurgical procedure, cutting tissue and/or cauterizing blood vessels with the electrode tip 112 will produce smoke. Additionally, cutting tissue will also cause bleeding and/or expose other bodily fluids. For a variety of reasons, it can be desirable to remove the smoke and bodily fluids from the surgical site. These reasons may include, but are not limited to, maintaining visibility of the surgical site, reducing exposure to the smoke, and the like. Accordingly, in a typical electrosurgical operating environment, a surgeon may use the electrosurgical instrument 104 to cut tissue and/or cauterize blood vessels and one or more other operating room personnel may use the smoke evacuation hand piece 118 and the fluid evacuation hand piece 126 to remove smoke and fluids from the surgical site.

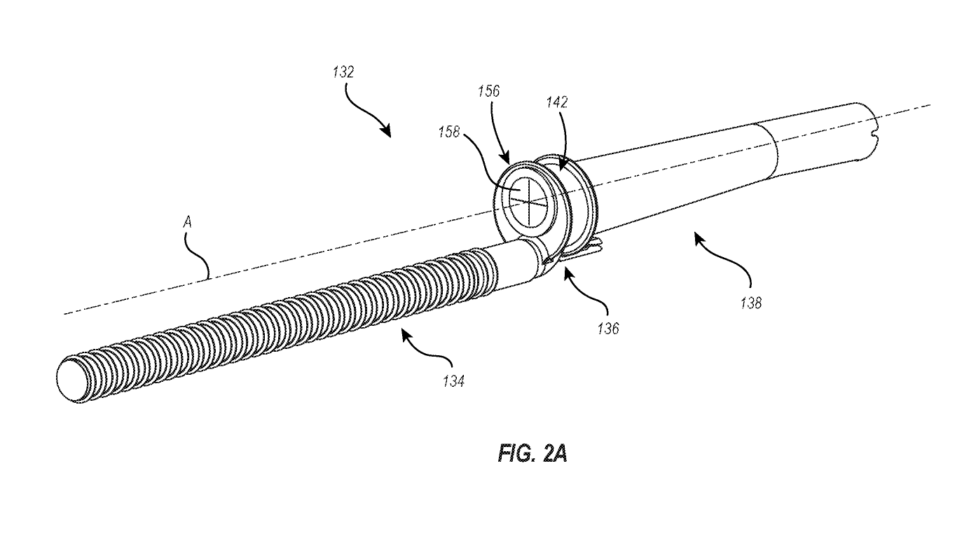

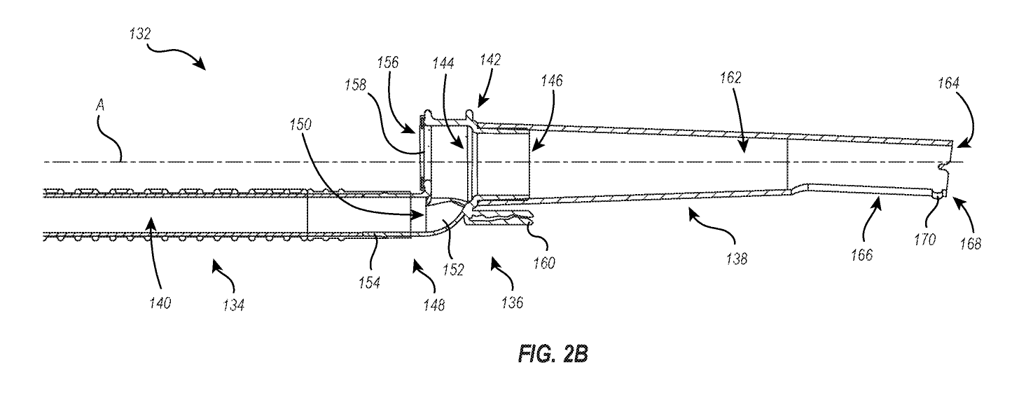

While the electrosurgical operating environment of FIG. 1 provides the various functions (e.g., cutting, cauterizing, smoke and fluid evacuation) needed during an electrosurgical procedure, the number of discrete instruments and associated personnel employed to perform these functions can create a crowded operating environment. To reduce the crowding at and around the surgical site, some of the components used at the surgical site may be combined, selectively or permanently, so that fewer discrete instruments are needed at the surgical site and fewer personnel are needed around the surgical site. FIGS. 2A-6B illustrate various example embodiments of such smoke and fluid evacuation devices that may be selectively or permanently combined to remove smoke and/or fluid from a surgical site.

FIGS. 2A and 2B illustrate an example embodiment of a smoke evacuation device 132 that is configured to evacuate smoke away from a surgical site. FIG. 2A is a rear perspective view of the smoke evacuation device 132 and FIG. 2B is a cross-sectional view thereof. Although the smoke evacuation devices disclosed herein are described as being able to evacuation or remove smoke from a surgical site, it will be appreciated that these devices may be able to evacuation other airborne contaminants away from a surgical site. Similarly, while the devices disclosed herein are described as being used in a surgical environment, this is merely exemplary and these devices may be used in other settings.

Generally, the smoke evacuation device 132 includes a hose 134, a hub 136, and a nozzle 138. The hose 134 includes a lumen 140 therethrough. A proximal end of the hose 134 may be in fluid communication with a pump (e.g., pump 120, FIG. 1) and a distal end of the hose 134 may be selectively or permanently attached to the hub 136. A negative pressure generated by the pump may be communicated to the hub 136 via the lumen 140 in the hose 134.

The hub 136 may be configured to be held by a user. Thus, the hub 136 may also be referred to as a hand piece. In the illustrated embodiment, the hub 136 includes a main body 142 with an interior cavity 144. The main body 142 includes a distal opening 146 that opens into the interior cavity 144. A longitudinal axis A of the main body 142 may extend through the distal opening 146 and/or be centered therein. The main body 142 also includes a hose mount 148 to which the hose 134 may be selectively or permanently connected. The hose mount 148 includes a lumen 150 therethrough. The lumen 150 is configured to enable fluid communication between the lumen 140 in the hose 134 and the interior cavity 144 in the main body 142.

In the illustrated embodiment, the hose mount 148 is laterally offset from the longitudinal axis A of the main body 142. The hose mount 148 may include an angled portion 152 and a straight portion 154. The angled portion 152 may extend at least partially laterally from the main body 142 and the straight portion 154 may extend proximally relative to the main body 142. In some embodiments, the straight portion 154 may extend generally parallel to the longitudinal axis A of the main body 142. In other embodiments, the straight portion 154 may extend perpendicular or at another angle relative to the longitudinal axis A.

The hub 136 also includes a port 156 in the main body 142. The port 156 includes a valve 158 that is configured to selectively open and close/seal the port 156. In the illustrated embodiment, the port 156 is disposed on a proximal side of the main body 142 and the longitudinal axis A passes therethrough. As discussed in greater detail below, the port 156 and valve 158 may be configured to have a fluid evacuation device inserted therethrough or removed therefrom.

The hub 136 also includes a retention feature 160. The retention feature 160 may be a clip, hook, or other element that is configured to be selectively secured to an element in an operating environment. For instance, the retention feature 160 may be configured to be secured to a surgical drape to hold the smoke evacuation device 132 in a desired position at or near a surgical site. Using the retention feature 160 to secure the smoke evacuation device 132 in place may allow for fewer hands to be around the surgical site, thereby creating a less crowded surgical area.

The nozzle 138 is connected to or integrally formed with the main body 142. The nozzle 138 extends distally from the main body 142. The nozzle 138 includes a lumen 162 extending therethrough. The lumen 162 is in fluid communication with the interior cavity 144 in the main body 142 of the hub 136. The nozzle 138 also includes a distal opening 164 that opens into the lumen 162. In some embodiments, the longitudinal axis A may extend through the lumen 162 and/or the distal opening 164.

In the illustrated embodiment, the distal opening 164 is in a terminal end of the nozzle 138 and opens in a distal direction. In other embodiments, the distal opening 164 may include one or more openings in the terminal end of the nozzle 138. In still other embodiments, the distal opening 164 may include one or more openings disposed about a circumference or other perimeter of the nozzle 138 and open radially.

As shown in FIG. 2B, the nozzle 138 may also include a slot 166 in a sidewall thereof. The slot 166 may extend proximally from the distal opening 164 along a portion of the length of the nozzle 138. An open channel 168 may be formed between the distal opening 164 and the slot 166. The slot 166 and/or channel 168 may include one or more retention features 170. As will be discussed in greater detail below, the slot 166, channel 168, and retention features 170 may allow for the positioning of a fluid evacuation device in one or more desired positions.

Smoke may be drawn into the lumen 162 in the nozzle 138 through the distal opening 164 and/or the slot 166. The smoke may pass through the lumen 162, the interior cavity 144, the lumen 150, and the lumen 140 to convey the smoke away from a surgical site. Thus, the opening 164, the slot 166, the lumen 162, the cavity 144, the lumen 150, and the lumen 140 may form a smoke evacuation pathway through the smoke evacuation device 132.

The smoke evacuation device 132 may also include one or more controls for governing the evacuation of smoke therethrough. For instance, the hub 136 may include one or more user inputs that are configured to start or stop the evacuation of smoke. In some embodiments, the one or more user inputs may be in communication with the associated pump. Activation of one or more of the user inputs on the hub 136 may relay signals to the associated pump that cause the pump to turn on to evacuate smoke or turn off when smoke evacuation is not needed. In other embodiments, the hose 134, hub 136, and/or nozzle 138 may include one or more valves that can be selectively opened or closed to allow for smoke evacuation or not.

Before proceeding further, it will be noted that the specific configuration of the smoke evacuation device 132 shown and described herein is merely exemplary. The smoke evacuation device 132 may be altered and still fall within the scope of the present disclosure. By way of example, the orientation and/or relative positioning of the hose mount 148 and the port 156 may be altered such that they are not positioned adjacent to one another or such that either or both of them do not extend or open proximally. Similarly, the shape and configuration of the nozzle 138 may vary from one embodiment to another. Still further, while the longitudinal axis A is illustrated as extending through the cavity 144 and port 156 in the main body 142 and the lumen 162 and opening 164 in the nozzle 138, this is only one example. In other embodiments, the longitudinal axis A may only extend through the cavity 144, and the port 156, the lumen 162, and/or the opening 164 may be positioned and/or oriented such that the longitudinal axis A does not extend therethrough.

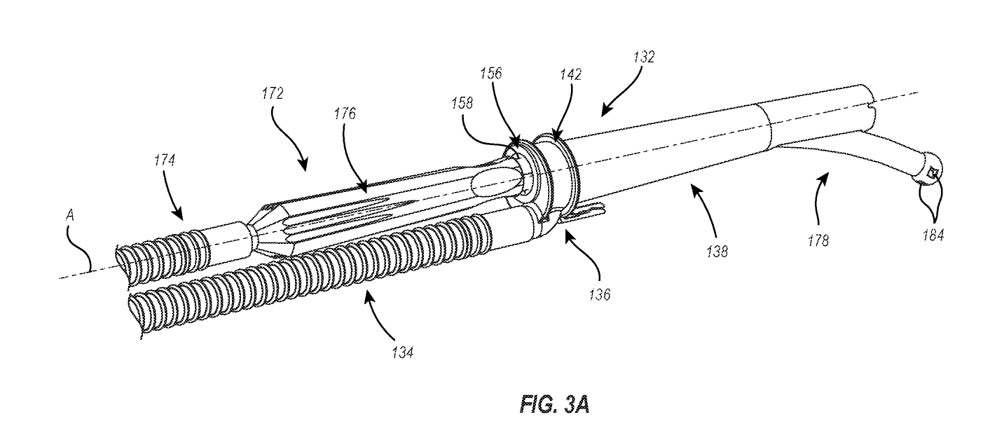

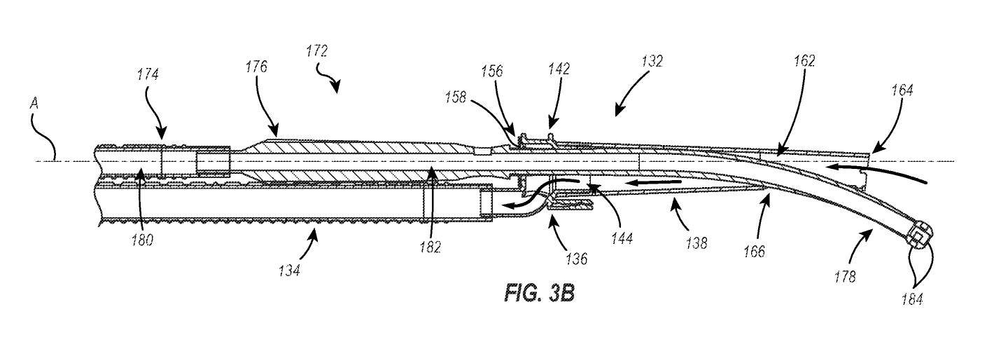

Attention is now directed to FIGS. 3A and 3B, which illustrate perspective and cross-sectional views of the smoke evacuation device 132 with a fluid evacuation device 172 associated therewith. The fluid evacuation device 172 includes a hose 174, a hand piece 176, and a nozzle 178. The fluid evacuation device 172 may be configured to evacuate fluid (e.g., blood or other bodily fluids) away from a surgical site. In some embodiments, the fluid evacuation device 172 may be a Yankauer device.

A proximal end of the hose 174 may be in fluid communication with a pump (e.g., pump 128, FIG. 1) and a distal end of the hose 174 may be selectively or permanently attached to the hand piece 176. The hose 174 may have a lumen 180 extending therethrough. A negative pressure generated by the pump may be communicated to the hand piece 176 via the lumen 180 in the hose 174.

The hand piece 176 may be configured to be held by a user during use of the fluid evacuation device 172. For instance, the hand piece 176 may be sized and configured to be comfortably held by a user’s hand.

The hand piece 176 and the nozzle 178 may be integrally formed or permanently or selectively connected together. A lumen 182 extends through the hand piece 176 and the nozzle 178. The lumen 182 is in fluid communication with the lumen 180 in the hose 174. The nozzle 178 includes one or more openings 184 at the distal end thereof. In the illustrated embodiment, the one or more openings 184 include an opening in the terminal end of the nozzle 178 that opens distally. Additionally, the one or more openings 184 include a plurality of openings disposed about the circumference or outer perimeter of the nozzle 178 and which open radially.

Fluid may be drawn into the lumen 182 in the hand piece 176/nozzle 138 through the one or more openings 184. The fluid may pass through the lumen 182 and into the lumen 180 in the hose 174 to convey the fluid away from a surgical site.

The fluid evacuation device 172 may also include one or more controls for governing the evacuation of fluid therethrough. For instance, the hand piece 176 may include one or more user inputs that are configured to start or stop the evacuation of fluid. In some embodiments, the one or more user inputs may be in communication with the associated pump. Activation of one or more of the user inputs on the hand piece 176 may relay signals to the associated pump that cause the pump to turn on to evacuate fluid or turn off when fluid evacuation is not needed. In other embodiments, the hose 174, hand piece 176, and/or nozzle 178 may include one or more valves that can be selectively opened or closed to allow for fluid evacuation or not.

As shown in FIGS. 3A and 3B, the fluid evacuation device 172 may be associated with the smoke evacuation device 132 to create a combination smoke and fluid evacuation device. More specifically, the fluid evacuation device 172 and the smoke evacuation device 132 may be connected together such that they can be held in a single hand of a user. In some embodiments, when so associated, the distal ends of the nozzles 138, 178 are positioned relatively close to one another such that smoke and fluid in close proximity to one another can be readily evacuated through the appropriate nozzle 138, 178 with relatively limited movement of the combined device.

The smoke evacuation device 132 and the fluid evacuation device 172 may be associated with one another in a variety of ways. For instance, FIGS. 3A and 3B illustrate one example manner for associating the two devices. In the illustrated embodiment, the nozzle 178 of the fluid evacuation device 172 is inserted through the port 156 and extends through the cavity 144 of the hub 136 and through the lumen 162 and out of either the opening 164 or the slot 166 in the nozzle 138. When so positioned, at least a portion of the nozzle 178 is disposed within the smoke evacuation pathway through the smoke evacuation device 132.

The dimensions of the smoke evacuation pathway through which the nozzle 178 extends and the exterior dimensions of the nozzle 178 may be sized to allow for smoke to flow around the nozzle 178 and through the smoke evacuation pathway, as indicated by the arrows in FIG. 3B. For instance, the cavity 144, lumen 162, opening 164, and, optionally, the slot 166, may have dimensions that are larger than the exterior dimensions of the nozzle 178. As a result, the nozzle 178 will not fill the entirety of the smoke evacuation pathway or block a block a path therethrough. Rather, while the nozzle 178 may occupy a portion of the smoke evacuation pathway, there can be sufficient area within the smoke evacuation pathway and around the nozzle 178 to allow for smoke to flow through the smoke evacuation pathway and around or past the nozzle 178.

The valve 158 associated with the port 156 may selectively open to allow for the nozzle 178 to extend therethrough. The valve 158 may be configured to seal around the outer surface of the nozzle 178 to limit or prevent smoke from escaping from the hub 136 through the port 156.

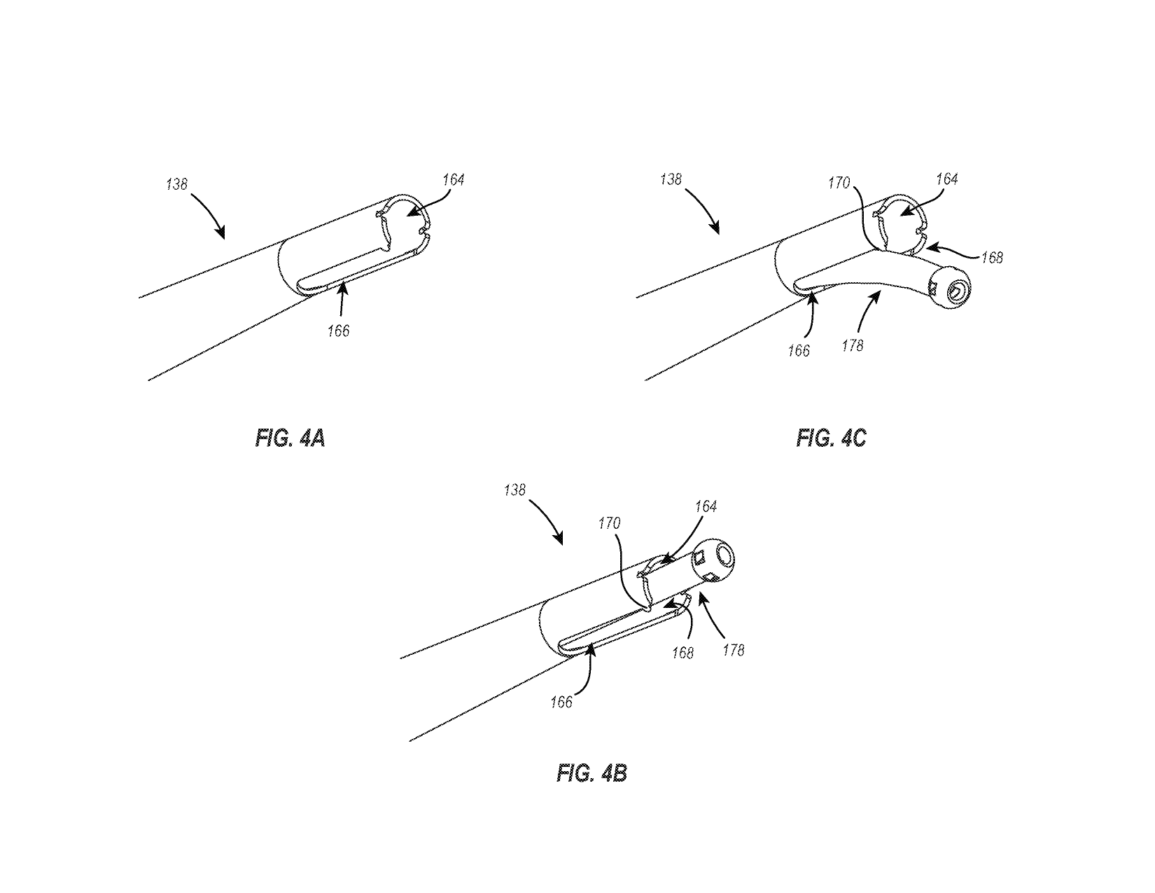

Attention is now directed to FIGS. 4A-4C, which illustrate the distal end of the nozzle 138. Specifically, FIG. 4A illustrates the distal end of the nozzle 138 alone, FIG. 4B illustrates the nozzle 138 with the nozzle 178 extending out of the distal opening 164 thereof, and FIG. 4C illustrates the nozzle 138 with the nozzle 178 extending of the slot 166 thereof.

As shown in FIG. 4A and noted above, the distal end of the nozzle 138 includes an opening 164 and an optional slot 166. Smoke may be drawn into the opening 164 and/or the slot 166 to evacuate the smoke. When the smoke evacuation device 132 is used apart from the fluid evacuation device 172, the opening 164 and the slot 166 may be open and unobstructed, as shown in FIG. 4A.

When the smoke evacuation device 132 is used in conjunction with the fluid evacuation device 172, the nozzle 178 may extend out of the nozzle 138 in one or more different configurations. For instance, as shown in FIG. 4B, the nozzle 178 may extend relatively straight out of the nozzle 138 through the opening 164 in the distal end of the nozzle 138. In such an embodiment, the opening 164 may be large enough to allow for the distal end of the nozzle 178 to pass therethrough. Additionally, in some embodiments, such as that shown in FIG. 4B, the opening 164 may be large enough to have the nozzle 178 extend therethrough while also allowing smoke to be drawn into the nozzle 138 around the nozzle 178. FIG. 4B uses arrows to illustrate smoke being drawn through the opening 164 and around the nozzle 178. In this configuration, the distal ends of the nozzles 138, 178 are generally aligned with one another. This may be desirable to reduce the overall profile of the combined smoke and fluid evacuation device

In some embodiments, it may be desirable to have the smoke and fluid evacuations be directed in different directions. FIG. 4C illustrates one example manner of accomplishing this desire. As can be seen, the nozzle 178 extends partially through the nozzle 138. However, instead of extending straight out of the nozzle 138 through the opening 164, the nozzle 178 is curved or bent and extends out of the nozzle 138 through the slot 166. The nozzle 178 may be flexible enough to allow for such bending without crimping or blocking the lumen 182 therethrough.

In some embodiments, the nozzle 178 is inserted through the nozzle 138 so that the distal end of the nozzle 178 extends out of the opening 164 in the nozzle 138, as shown in FIG. 4B. Thereafter, the nozzle 178 may bent and moved through the channel 168 and into the slot 166, as shown in FIG. 4C.

The one or more retention features 170 may be configured to hold the nozzle 178 in the slot 166 and prevent the nozzle 178 from unbending and moving back through the channel 168 into the opening 164. The one or more retention features 170 may include one mor more protrusions that extend into the channel 168 and/or the slot 166. The distance between the protrusions may be smaller than an outer dimension of the nozzle 178. The nozzle 178 may be formed of a compressive or flexible enough material to allow for the nozzle 178 to be intentionally squeezed through the protrusions (e.g., by a user when moving the nozzle 178 between the opening 164 and the slot 166). However, the protrusions may be close enough together to prevent the nozzle move unintentionally moving thereby.

In some embodiments, the slot 166 may include one or more additional retention features disposed along the length thereof. The one or more additional retention features may be configured to allow for the nozzle 178 to extend out of the nozzle 138 (through the slot 166) at various different positions along the length of the slot 66.

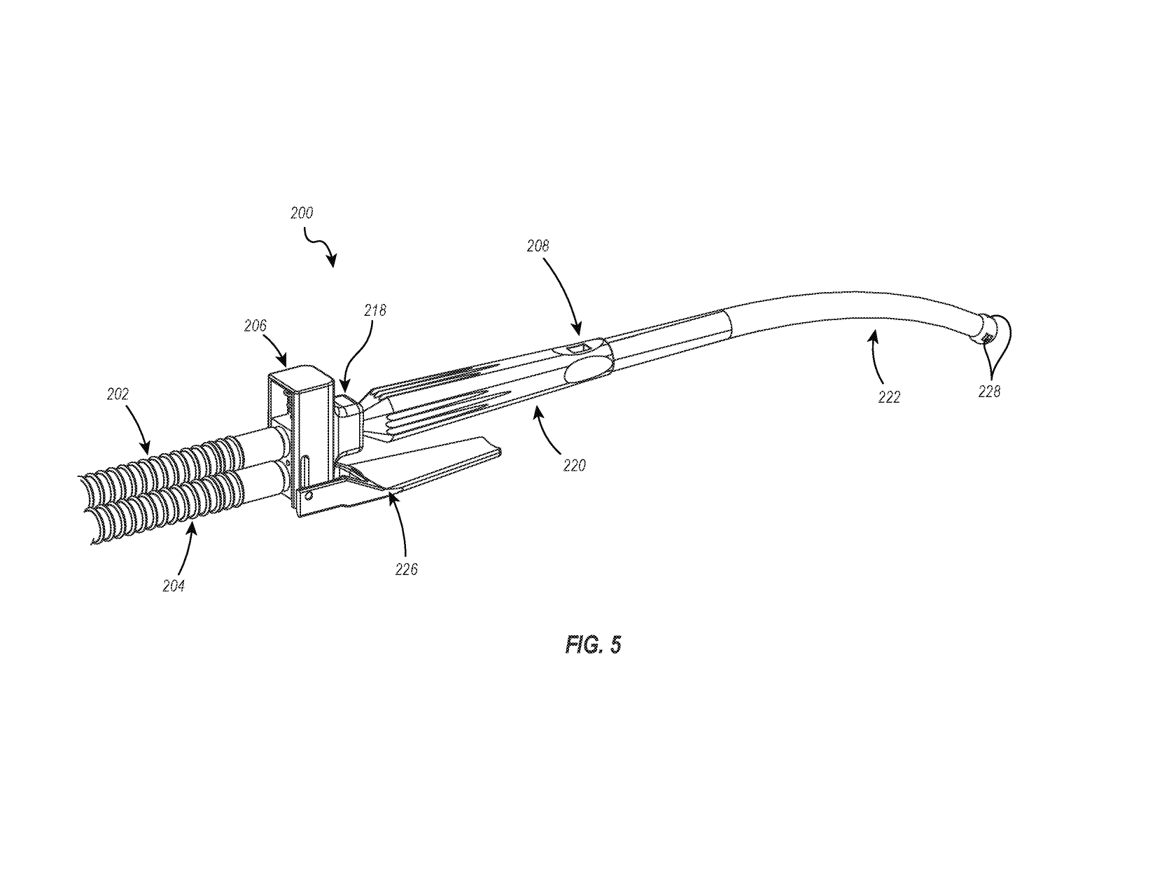

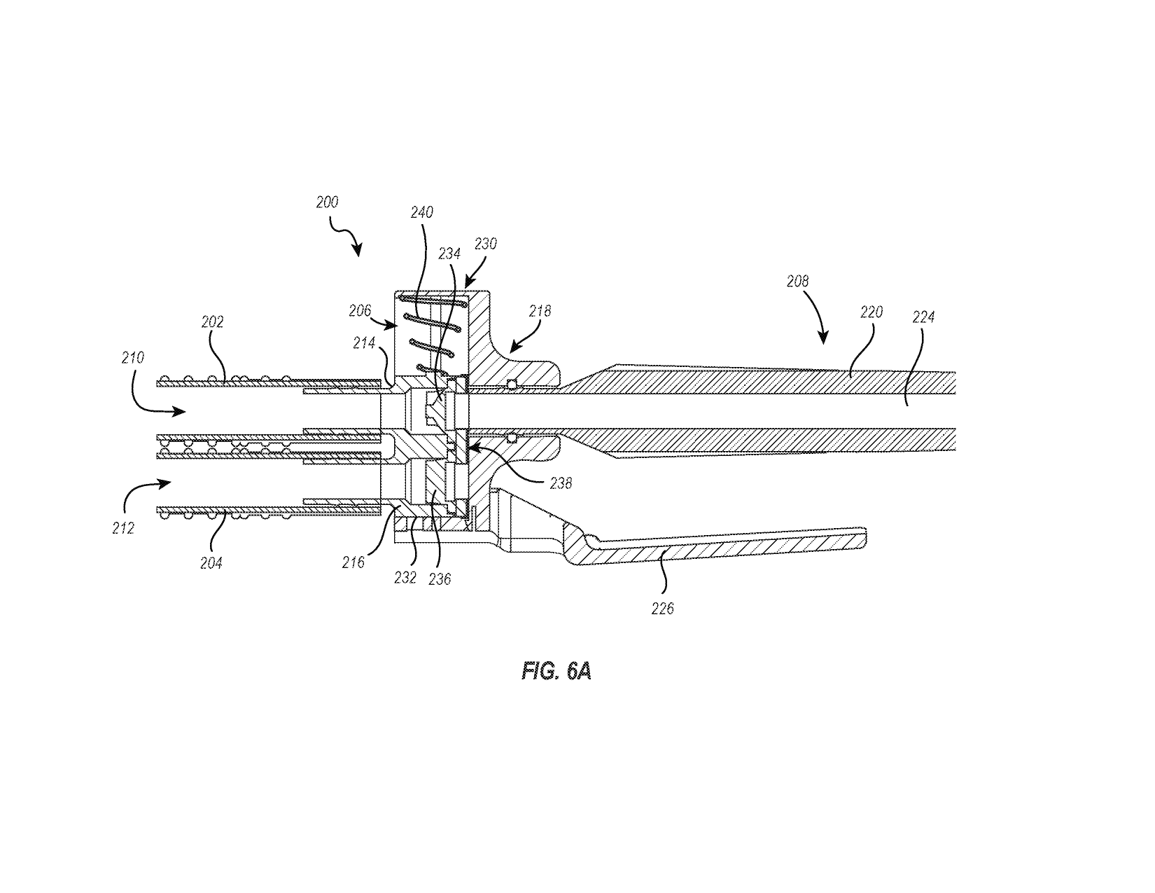

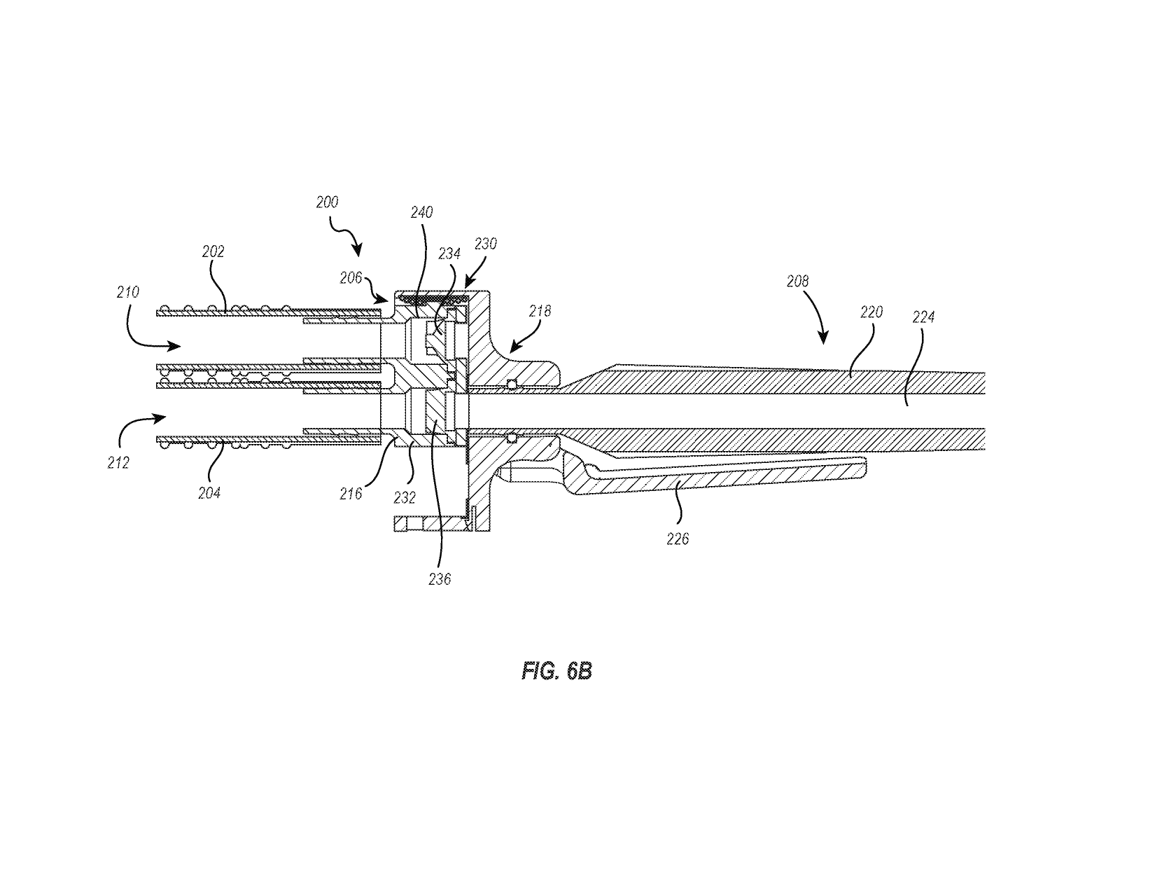

Attention is now directed to FIGS. 5-6B, which illustrate an example embodiment of a combination smoke and fluid evacuation device 200. The device 200 may be configured to alternatively evacuate smoke and fluids from a surgical site. Generally, the device 200 includes a smoke evacuation hose 202, a fluid evacuation hose 204, a hub 206, and a wand 208. The hub 206 may also be referred to as a switching component.

A proximal end of the smoke evacuation hose 202 may be connected to a pump (e.g., pump 120, FIG. 1) and a proximal end of the fluid evacuation hose 204 may be connected to a pump (e.g., pump 128, FIG. 1). In some embodiments, the hoses 202, 204 may be connected to the same pump. Whether connected to separate pumps or the same pump, the pump(s) can be configured to generate negative pressures that are communicated through the hoses 202, 204. More specifically, the negative pressures can be communicated through lumens 210, 212 extending respectively through the hoses 202, 204.

Distal ends of the hoses 202, 204 may be selectively or permanently connected to the hub 206. The hub 206 includes a first hose mount 214 (also referred to as a smoke evacuation hose mount) to which the smoke evacuation hose 202 is or can be connected. The hub 206 also includes a second hose mount 216 (also referred to as a fluid evacuation hose mount) to which the fluid evacuation hose 204 is or can be connected. The negative pressures communicated through the hoses 202, 204 can be communicated to the hub 206 via the hose mounts 214, 216.

The hub 206 also includes a wand mount 218 to which the wand 208 is or can be selectively or permanently connected. The wand 208 includes a hand piece portion 220 and a nozzle 222. A lumen 224 extends through the wand 208. The distal end of the wand 208 includes one or more openings 228. The one or more openings 228 may include an opening in a terminal end of the wand 208 that opens distally. The one or more openings 228 may also include one or more opening disposed around a circumference or perimeter of the nozzle 222 and which open radially.

The hub 206 may be configured to selectively and alternately communicate the negative pressure from hoses 202, 204 to the wand 208 (and particularly the lumen 224 therein). For instance, in a first configuration as shown in FIGS. 5 and 6A, the hub 206 may be configured to enable fluid communication between the hose 202 and the wand 208 while preventing fluid communication between the hose 204 and the wand 208. In the first configuration, the negative pressure from the smoke evacuation hose 202 may be communicated through the hub 206 and the lumen 224 in the wand 208 to draw smoke into and through the device 200 (including through the smoke evacuation hose 202).

In a second configuration as shown in FIG. 6B, the hub 206 may be configured to enable fluid communication between the hose 204 and the wand 208 while preventing fluid communication between the hose 202 and the wand 208. In the second configuration, the negative pressure from the fluid evacuation hose 204 may be communicated through the hub 206 and the lumen 224 in the wand 208 to draw fluid into and through the device 200 (including through the fluid evacuation hose 204).

As shown in the FIGS. 5-6B, the hub 206 may include an input mechanism 226. The input mechanism 226 may be configured to move the hub 206 between the first configuration and the second configuration. The input mechanism 226 may also be configured to communicate with the smoke and/or fluid evacuation pumps to activate or deactivate the pumps.

For instance, movement of the input mechanism 226 to the first configuration may cause a signal to be sent to the smoke evacuation pump that causes the smoke evacuation pump to be activated and generate a negative pressure that is communicated through the hose 202, the hub 206, and the wand 208. Movement of the input mechanism 226 to the first configuration may also cause a signal to be sent to the fluid evacuation pump that causes the fluid evacuation pump to be deactivated so that a negative pressure is not communicated through the hose 204.

Similarly, movement of the input mechanism 226 to the second configuration may cause a signal to be sent to the fluid evacuation pump that causes the fluid evacuation pump to be activated and generate a negative pressure that is communicated through the hose 204, the hub 206, and the wand 208. Movement of the input mechanism 226 to the second configuration may also cause a signal to be sent to the smoke evacuation pump that causes the smoke evacuation pump to be deactivated so that a negative pressure is not communicated through the hose 202.

FIGS. 6A and 6B illustrate one example embodiment of a mechanism that can be used to switch the hub 206 between the first and second configurations. In the illustrated embodiment, the hub 206 includes a housing 230 and a valve carriage 232. The valve carriage 232 is slidably disposed within the housing 230 between a first position as shown in FIG. 6A and a second position as shown in FIG. 6B.

The valve carriage 232 includes a first valve 234 and a second valve 236. The first hose mount 214 is connected to the valve carriage 232 and is in fluid communication with the first valve 234. The second hose mount 216 is connected to the valve carriage 232 and is in fluid communication with the second valve 236. The first and second valves 234, 236 may be selectively opened upon an application of a negative pressure applied thereto (e.g., from the associated hose 202, 204).

Movement of the valve carriage 232 within the housing 230 between the first and second positions will cause the first valve 234 or the second valve 236 to be aligned with the wand mount 218. For instance, as shown in FIG. 6A, when the valve carriage 232 is in the first position, the first valve 234 is aligned with the wand mount 218. A negative pressure communicated through the hose 202 and the first hose mount 214 will be communicated through the first valve 234 and the lumen 224 in the wand 208. In this configuration, smoke can be drawn into the wand 208 and evacuated away from a surgical site through the wand 208, hub 206, and hose 202.

When fluid evacuation is desired, the valve carriage 232 may be moved from the first position to the second position, as shown in FIG. 6B. When the valve carriage 232 is in the second position, the second valve 236 is aligned with the wand mount 218. A negative pressure communicated through the hose 204 and the second hose mount 216 will be communicated through the second valve 236 and the lumen 224 in the wand 208. In this configuration, fluid can be drawn into the wand 208 and evacuated away from a surgical site through the wand 208, hub 206, and hose 204.

The housing 230 and valve carriage 232 may include one or more features that facilitate smooth and consistent movement of the valve carriage 232 between the first and second positions. For instance, the housing 230 and valve carriage 232 may include mating or corresponding surfaces (e.g., tracks, rails, etc.) that guide and control the movement of the valve carriage 232 within the housing 230.

The hub 206 may also include a seal 238 disposed between the valve carriage 232 and the wand mount 218. In the illustrated embodiment, the seal 238 is mounted on the valve carriage 232 and extends around the first and second valves 234, 236. In other embodiments, the seal 238 may be mounted to the interior of the housing 230. In any event, the seal 238 may be configured to create a fluid and airtight seal between the first valve 234 and the wand mount 218 when the valve carriage 232 is in the first position and a fluid and air right seal between the second valve 236 and the wand mount 218 when the valve carriage 232 is in the second position.

The hub 206 may also include a biasing element 240 that is configured to bias the valve carriage 232 to either the first or second position. In the illustrated embodiment, the biasing element 240 comprises a spring disposed between the valve carriage 232 and an interior surface of the housing 230. The spring may urge the valve carriage 232 toward the first position, so that the device 200 defaults to a smoke evacuation configuration. In other embodiments, the biasing element 240 may be arranged to urge the valve carriage 232 towards the second position, so that the device 200 defaults to a fluid evacuation configuration.

As noted above, the hub 206 also includes the input mechanism 226 that can be used to move switch the device 200 between the smoke and fluid evacuation configurations. In the illustrated embodiment, the input mechanism 226 comprises a lever or handle that is connected to the valve carriage 232. Moving the input mechanism 226 relative to the housing 230 causes the valve carriage 232 to move within the housing 230 as described. In use, a user may hold the hand piece portion 220 to place the distal end of the nozzle 222 in a desired location. With the same hand or an additional hand, the user may engage the input mechanism 226 to move the input mechanism 226 relative to the housing 230, and thereby switch the device from the smoke evacuation mode to the fluid evacuation mode. The user may also release the input mechanism 226 and the biasing element 240 will cause the valve carriage 232 to move to the second position to switch the device 200 to the smoke evacuation mode.

While the embodiment of FIGS. 5-6B have been shown and described in particular detail, it will be appreciated that it is provided merely by way of example. As contemplated herein, a combination smoke and fluid evacuation device may include a smoke evacuation hose, a fluid evacuation hose, a wand, and a device for selectively switching the fluid communication between the wand and the two hoses. The switching device may take any number of forms, including valves or gates that can be selectively opened and closed to switch between smoke evacuation and fluid evacuation.

It is understood that the features of the above-described embodiments are not exclusive to one another. Rather, one of ordinary skill in the art will recognize that the described features can be combined and/or modified as may be needed or desired.

While the embodiments disclosed herein have been directed to smoke and fluid evacuation device used in an electrosurgical operating environment, the present invention is not intended to be limited only to operating environment. Rather, the present invention is broadly directed to any devices or combinations of devices that can be used to alternately evacuate smoke/airborne contaminants and fluids as described herein. Examples of such devices may include, but are not limited to, suction handles (e.g., Yankauer), vacuum hand pieces, surgical suction instruments (e.g., liposuction cannulas), surgical suction cannulas, and the like.

According to one embodiment, a smoke evacuation device comprises: a hub comprising an interior cavity, a port that is configured to provide selective access to the interior cavity, a distal opening, and a hose mount in fluid communication with the interior cavity; and a nozzle extending from the hub, the nozzle having a lumen extending therethrough that is in fluid communication with interior cavity via the distal opening in the hub, the nozzle comprising one or more opening in a distal end thereof, the port, interior cavity, and nozzle being configured to have a fluid evacuation device selectively positioned therethrough such that a portion of the fluid evacuation device extends out of the port and a portion of the fluid evacuation device extends out of the distal end of the nozzle.

In some embodiments, the port comprises a valve that is configured to close the port when the fluid evacuation device is not positioned therethrough.

In some embodiments, the valve is configured to create a seal around the fluid evacuation device when the fluid evacuation device is positioned to extend therethrough.

In some embodiments, the smoke evacuation device comprises a longitudinal axis that extends through the cavity, port, and nozzle.

In some embodiments, the hose mount is laterally offset from the longitudinal axis.

In some embodiments, the hub comprises a retention feature that is configured to selectively secure the smoke evacuation device to another component.

In some embodiments, the one or more openings in the nozzle comprise a distally facing opening in a terminal end of the nozzle.

In some embodiments, the nozzle comprises a proximally extending slot formed in a side thereof, the slot being configured to have a portion of the fluid evacuation device extend therethrough.

In some embodiments, the distally facing opening and the slot are connected together by a channel, the channel being configured to allow for the portion of the fluid evacuation device to be selectively moved between the distally facing opening and the slot through the channel.

In some embodiments, a smoke evacuation hose is connected or connectable to the hose mount.

In one embodiment, combination smoke and fluid evacuation device for use during an electrosurgical procedure to evacuate smoke and fluid away from a surgical site comprises: a wand configured to be used to collect smoke and fluid from a surgical site; a smoke evacuation hose mount that is configured to be connected to a smoke evacuation hose; a fluid evacuation hose mount that is configured to be connected to a fluid evacuation hose; a switching component that is configured to enable selective fluid communication between the wand and alternately the smoke evacuation hose mount and the fluid evacuation hose mount.

In some embodiments, the wand comprises a lumen extending therethrough and one or more openings at a distal end thereof.

In some embodiments, the switching component comprises a housing.

In some embodiments, the housing comprises a wand mount that is configured to have the wand connected thereto.

In some embodiments, the switching component further comprises a valve carriage that is selectively movable within the housing between first and second positions.

In some embodiments, the valve carriage comprises a first valve associated with the smoke evacuation hose mount and a second valve associated with the fluid evacuation hose mount.

In some embodiments, the first valve is in fluid communication with the wand mount when the valve carriage is in the first position and the second valve is in fluid communication with the wand mount when the valve carriage is in the second position.

### Conclusion

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A smoke evacuation device comprising:
a hub comprising an interior cavity, a port that is configured to provide selective access to the interior cavity, a distal opening, and a hose mount in fluid communication with the interior cavity; and
a nozzle extending from the hub, the nozzle having a lumen extending therethrough that is in fluid communication with interior cavity via the distal opening in the hub, the nozzle comprising one or more openings in a distal end thereof,
the port, interior cavity, and nozzle being configured to have a fluid evacuation device selectively positioned therethrough such that a portion of the fluid evacuation device extends out of the port and a portion of the fluid evacuation device extends out of the distal end of the nozzle.

2. The smoke evacuation device of claim 1, wherein the port comprises a valve, the valve being configured to close the port when the fluid evacuation device is not positioned therethrough.

3. The smoke evacuation device of claim 2, wherein the valve is configured to create a seal around the fluid evacuation device when the fluid evacuation device is positioned to extend therethrough.

4. The smoke evacuation device of claim 1, wherein the smoke evacuation device comprises a longitudinal axis that extends through the cavity, port, and nozzle.

5. The smoke evacuation device of claim 4, wherein the hose mount is laterally offset from the longitudinal axis.

6. The smoke evacuation device of claim 1, wherein the hub comprises a retention feature that is configured to selectively secure the smoke evacuation device to another component.

7. The smoke evacuation device of claim 1, wherein the one or more openings in the nozzle comprise a distally facing opening in a terminal end of the nozzle.

8. The smoke evacuation device of claim 7, wherein the nozzle comprises a proximally extending slot formed in a side thereof, the slot being configured to have a portion of the fluid evacuation device extend therethrough.

9. The smoke evacuation device of claim 8, wherein the distally facing opening and the slot are connected together by a channel, the channel being configured to allow for the portion of the fluid evacuation device to be selectively moved between the distally facing opening and the slot through the channel.

10. The smoke evacuation device of claim 1, further comprising a smoke evacuation hose connected or connectable to the hose mount.

11.
A combination smoke and fluid evacuation device for use during an electrosurgical procedure to evacuate smoke and fluid away from a surgical site, the device comprising:
a wand configured to be used to collect smoke and fluid from a surgical site;
a smoke evacuation hose mount that is configured to be connected to a smoke evacuation hose;
a fluid evacuation hose mount that is configured to be connected to a fluid evacuation hose; and
a switching component that is configured to enable selective fluid communication between the wand and alternately the smoke evacuation hose mount and the fluid evacuation hose mount.

12. The combination smoke and fluid evacuation device of claim 11, wherein the wand comprises a lumen extending therethrough and one or more openings at a distal end thereof.

13. The combination smoke and fluid evacuation device of claim 11, wherein the switching component comprises a housing.

14. The combination smoke and fluid evacuation device of claim 13, wherein the housing comprises a wand mount that is configured to have the wand connected thereto.

15. The combination smoke and fluid evacuation device of claim 14, wherein the switching component further comprises a valve carriage that is selectively movable within the housing between first and second positions.

16. The combination smoke and fluid evacuation device of claim 15, wherein the valve carriage comprises a first valve associated with the smoke evacuation hose mount and a second valve associated with the fluid evacuation hose mount.

17. The combination smoke and fluid evacuation device of claim 16, wherein the first valve is in fluid communication with the wand mount when the valve carriage is in the first position and the second valve is in fluid communication with the wand mount when the valve carriage is in the second position.

18. A combination smoke and fluid evacuation device for use during an electrosurgical procedure to evacuate smoke and fluid away from a surgical site, the device comprising:
a wand configured to be used to collect smoke and fluid from a surgical site;
a smoke evacuation hose mount that is configured to be connected to a smoke evacuation hose;
a fluid evacuation hose mount that is configured to be connected to a fluid evacuation hose; and
a switching component that is configured to enable selective fluid communication between the wand and alternately the smoke evacuation hose mount and the fluid evacuation hose mount; the switching component comprising
a housing comprising a wand mount that is configured to have the wand connected thereto;
a valve carriage that is selectively movable within the housing between first and second positions, the valve carriage comprising a first valve associated with the smoke evacuation hose mount and a second valve associated with the fluid evacuation hose mount, the first valve being configured to be in fluid communication with the wand mount when the valve carriage is in the first position and the second valve being configured to be in fluid communication with the wand mount when the valve carriage is in the second position.

19. The combination smoke and fluid evacuation device of claim 18, wherein the wand comprises a lumen extending therethrough and one or more openings at a distal end thereof.

20. The combination smoke and fluid evacuation device of claim 18, wherein the valve carriage is biased to either the first position or the second position.
